(19) United States
(12) Patent Application Publication
(10) Pub. No.: US 2013/0122575 A1
(43) Pub. Date: May 16, 2013

(54) DEVISE TO PRODUCE ALCOHOL, BIO FUELS AND OTHER COMPOUNDS WITH A SEA BASED FERMENTOR

(71) Applicant: Panduranga Revankar Krishna Prasad, Bangalore (IN)

(72) Inventor: Panduranga Revankar Krishna Prasad, Bangalore (IN)

(21) Appl. No.: 13/261,572

(22) PCT Filed: Jul. 20, 2011
(86) PCT No.: PCT/IN2011/000480

§ 371 Date: Jan. 23, 2013

(30) Foreign Application Priority Data
Jul. 20, 2010 (IN) 2070/CHE/2010

(51) Int. Cl. C12M 1/107

(52) U.S. Cl. CPC C12M 21/04
USPC 435/287.1; 435/290.1

(57) ABSTRACT

A device, system, and method to produce alcohol and Bio fuels from bulky organic matter with a Sea based Fermentor with low cost per unit volume, formed from a plastic separator barrier which is flexible plastic film, a solar based distillation still with vacuum assist are disclosed. In one embodiment, a device includes a Fermentor coupled to a inlet channel made of plastic film separator, converted to Continuous process Fermentation channel, a Bio Gas digester to generate methane, a floating platform having engine, pumps, centrifuge and sterilization unit and a crane, a water treatment unit formed from plastic film is provided

### CLAIMS OF PRIORITY

This patent application claims priority from the Provisional Patent Application No. 2070/CHE/2010 filed on 20 Jul. 2010.

### FIELD OF TECHNOLOGY

This disclosure relates generally to technical fields of Alcohol and Bio Fuel production, and in one embodiment to a device to produce Alcohol used as a fuel with the devise comprising of a plastic separator barrier based flexible Fermentor in the sea, with floating Solar energy based Distillation still with vacuum assist, solid state fermentor as pre treatment to breakdown cellulose and lignin, a floating platform to carry out sterilization, product separation and storage and media removal for transfer from solid state fermentor to main Fermentor, open and closed type flexible film based channels with Isolator connector to transfer media, fresh water and spent matter back as fertilizer.

### BACKGROUND OF INVENTION

It is essential that the Alcohol and other organic Bio fuel like butenol production process consume less energy for production than the caloric value of the fuel produced and cost of production kept below the cost of fossil fuels for sustainable commercial production and use. This is possible only by having a low cost per unit volume of fermentor, which can be realized on the sea where the sea acts as a virtual chamber with plastic barrier film forming the separation between the organic Media and sea water. Solar energy based distillation with vacuum assist from wave energy reduces carbon footprint of production. Burning of cellulosic and lignin waste cuts the phosphorus and other nutrient cycle as the world food production will be limited by the availability of Phosphorus by mining which is a finite resource. so it is important to extract the fuel value of organic wastes and return the nutrients like phosphorus back to the biosphere as fertilizer.

The advantage of the present invention lies in the Flexible structures made of plastic with Leak detection system and multiplayer protective layers to prevent leakage thereby enabling large volume of fermentor chamber at low cost per unit volume.
Organic waste mainly containing cellulose and Lignin, Cellulosic and Ligin material being bulky need large volume at low costs for the slow fermentation process to yield fuels without consuming large amount of energy to produce the same. The present invention addresses this problem.
The main advantage of the invention is the very large volume of Fermentor at a low cost. The present invention addresses both the above requirement enabling bio fuel production from organic waste, together with the use of recycled plastics for structural strength. This devise, system and method is a True and Real Green technology needed to address the Global warming problem.

### SUMMARY

A device, system, and method to a device to produce Alcohol, Bio fuel and other Organic chemicals from cellulosic, ligin and organic matter using a plastic separator barrier based flexible Fermentor chamber in the sea. In one aspect, a device includes a Fermentor coupled to a inlet channel made of plastic film separator based channels to convey fresh water and media, the Fermentor is held by multi-chambered floats forming the upper periphery with a rigid frame covered with plastic film, a protection jacket being multi-chambered made from plastic film encloses the Fermentor to enable detection of leakage for taking up repair, an occurrence of leakage is detected through electrodes as sensors. A layer of netting is provided for protection from fish and other aquatic animals

In another aspect, the Media and fresh water are conveyed by channel made of plastic component may be an open type channel, and a closed type channel and the open type channel may be formed of flexible plastic film reinforced by fiber threads and polymeric fabric coated with plastic and of rubber sheet reinforced with fiber thread and metal cable for strength, held by multi-chambered floats with Isolator Connector being rigid having directional valve, and points for fixing plastic ropes for anchorage, and stabilization, and floats with detachable weights for wave management which may be reattached later and till that time held by separate floats. The plastic flexible film based channel is surrounded by leakage detection chambers with sensors, and the Closed type channels is formed of flexible plastic film reinforced with fiber threads, with Isolator Connector being rigid having directional valve, and points for fixing plastic ropes for anchorage, and stabilization, and floats with detachable weights for wave management which may be reattached later and till that time held by separate floats, and the plastic flexible film surrounded by leakage detection chambers with sensors. The open type, and closed type channel to have ‘U’ shaped connecting chamber made of rigid material with swivel joints, to allow the ships to cross the channel. The Closed channel is converted into a Fermentation chamber for Continuous process of fermentation by including a stirring mechanism powered by one of fluid pressure pneumatic and of sealed electric motor, a heat exchanger, a camera monitoring and sensors for process parameters pH, temperature, and other parameters required with provision for taking samples for testing.

The Fermentor may be held by multi-chambered plastic floats embedded with plastic ropes to provide hold for strong anchorage, and stabilization, and the Fermentor to rest on sea bed where it is shallow. The Fermentor may be made of plastic flexible film with reinforcing fiber thread and polymeric fabric coated with plastic. The plastic film is made of two layers including a strong thick outer layer made of recycled plastic, and an inner thin layer made of much thinner newly synthesized plastic to give better sealing.

In yet another aspect, stainless steel electrodes may be used in the multi-chambered wall which are connected by at least metallic wire embedded in plastic film and of conductive plastic to detect salt water leakage which leads to increased electrical conductivity detected by electronic system and thereby enable repair at the needed place.

The suction channel may be provided to the Fermentor to remove remaining spent media which is treated in a Bio gas Digestor coupled through a inlet channel made of plastic film separator to Fermentor, the spent media containing fresh water is allowed to decompose releasing methane, the digester chamber is held by multi-chambered floats forming the upper periphery, a protection jacket being multi-chambered made from plastic film encloses the storage chamber to enable detection of leakage for taking up repair, an occurrence of leakage is detected through electrodes as sensors, a plastic sheet is used to cover the storage chamber to collect methane gas, the plastic sheets slides over the floats, weights are attached to the plastic sheet to hold in tight position, and to apply pressure to allow the flow of methane gas, the methane gas is burnt after scrubbing, on the floating Platform in a engine to which is coupled a electric generator to generate power and operate pumps, and vacuum pump, a suction channel coupled to the Bio Gas digestion chamber is provided to remove digested organic matter which is pumped out and sent to coast for use as fertilizer, the channel runs parallel to supply channels. The remaining fresh water may be pumped into storage system formed of flexible plastic film reinforced by fiber thread, held by floats for water treatment sedimentation and filtration and with chemical treatment with at least chlorine, and used for the process with excess pumped to coast. The fresh water is treated in the sea by this device allowing fresh water treatment in the sea and the sent back to land for use.

In yet another aspect, the alcohol generated by fermentation is pumped from the Fementor to a solar powered distillation unit comprising a heater one of glass tube having heat exchanger surrounded by vacuum enclosed in a floating plastic container with glass sheet cover on top to trap solar radiation and prevent heat from escaping and one of heat exchanger enclosed in a floating plastic container with glass sheet cover on top to trap solar radiation and prevent heat from escaping, with vapour of alcohol condensing in the floating retort having balancer floats and condenser which is cooled using cold water circulated by pump support in a closed circuit so that minimum pumping energy is used. The evaporation is assisted by vacuum derived from pumps operating on tidal and sea waves and from vacuum pump on platform. The solar powered distillation unit is used to generate fresh water and hot water for generating steam for sterilization.

In yet another aspect, a floating platform having engine and pumps, a sterilization unit with steam generated using hot water from solar distillation unit with supplementary heating using one of bio gas from bio gas generator and a tank for chemical sterilization and UV radiation source, a storage tank for products and media, a centrifuge to separate products from the media, a crane to lift solid state fermentor to remove pretreated media, disintegrate for sending it to Fermentor for breakdown to alcohol, a Microprocessor based control system connected to sensor and camera signal outputs from Fermentor and distillation unit for interpretation of the process and provide signal for actuation of motors and valves by means of dedicated software, and provide a resting place for workers.

A solid state fermentor made of flexible plastic film held by rigid End connectors made of plastic material, with inlet and outlet channels to connect to supply channels to add media which is sterilized by chemicals and heat, a matrix of plastic helps to disintegrate the pretreated media, a pipe with holes to force fresh water to enable disintegration of the fungus grown media. The solid state fermentor is used to produce edible mushrooms on the platform as a by product of the devise.

The device, systems, and methods disclosed herein may be implemented in any means for achieving various aspects, and may be executed in a form of a machine-readable medium embodying a set of instructions that, when executed by a machine, cause the machine to perform any of the operations disclosed herein. Other features will be apparent from the accompanying drawings and from the detailed description that follows.

### BRIEF DESCRIPTION OF THE DRAWINGS

Example embodiments are illustrated by way of example and not limitation in the figures of the accompanying drawings, in which like references indicate similar elements and in which:

FIG. 1 is a system view of entire devise illustrating processing of media in Main fermentor conveyed through separator barrier channel, pretreatment in solid state fermentor, platform for sterilization and transfer of pretreated media from solid state fermentor and storage and control, Solar energy base distillation still with vacuum assist, bio gas digestor according to one embodiment.

FIG. 2 is a cross section view of the Main Fermentor (chamber) and its components, according to one embodiment.

FIG. 3 is a cross sectional view of Continuous Fermentation process Channel, according to one embodiment.

FIG. 4 is a cross section view of the open channel, according to one embodiment.

FIG. 5 is a top view of the open channel, according to one embodiment

FIG. 6 is a cross section view of the closed channel, according to one embodiment.

FIG. 7 is a side view of the closed channel, according to one embodiment

FIG. 8 is a side view of the open channel, according to one embodiment.

FIG. 9 is a side view of the closed channel, according to one embodiment.

FIG. 10 is a cross section view of a towing boat, according to one embodiment.

FIG. 11 is a system view of bellow chamber with isolator to cross ships, according to one embodiment.

FIG. 12 is a system view of a ‘U’ shaped connectivity chamber, according to one embodiment.

Other features of the present embodiments will be apparent from the accompanying drawings and from the detailed description that follows.

### DETAILED DESCRIPTION

A device, system and method to produce Alcohol, Bio fuels and other organic compounds in a sea based fermentor. Although the present embodiments have been described with reference to specific example embodiments, it will be evident that various modifications and changes may be made to these embodiments without departing from the broader spirit and scope of the various embodiments.

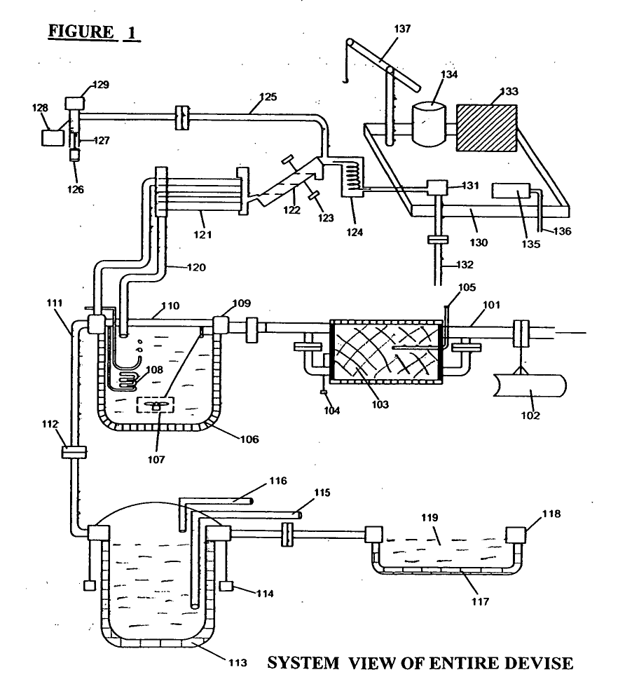

FIG. 1 is a system view of the entire devise with floating units on the sea illustrating pretreatment in solid state fermentor, fermentation in main fermentor, distillation in the solar based still and processing of spent waste in Bio gas digestor to produce methane, Platform to carry out sterilization, storage and control according to one embodiment. Particularly, FIG. 1 illustrates a media and fresh water conveyed by channel 101 from coast with Isolater connector with attached damper 102, a solid state fermentor 103 with attachable and detachable weight 104, tube with holes to apply water at force to disintegrate pretreated media with the help of one of fungus 105, main fermentor 106 with stirrer 107, heat exchanger 108, the fermentor having a framework on top 110, a channel (pipe or tube) 111 to transfer spent media to the Bio gas Digestor 113, channel (tube) 120 to transfer fermented media to solar based distillation unit to recover alcohol, the solar heater 121 with glass tubes held by frame, the evaporated alcohol vapour passed to the distillation resort 122 having balancer floats 123 and condensor 124 which has cooling from cold water drawn from deep sea by pump 131 through pipe (channel) 132, the distillation process is assisted by vacuum through vacuum line 125 which derives vacuum from one by pumps operated by tidal waves on cylinder 127 connected to float 129, having piston 127 connected to weight 126 which is anchoured to sea floor, the devise being connected to a damper 128, according to one embodiment.

The Media and Fresh water channel 100 (e.g., the open type channel, and the closed type channel) may be used to convey the Media and fresh water to Fermentor chamber. The Media 102 may be cellulosic and Lignin waste, heat treated corn, cellosic waste treated by heat and enzymes on the coast based heater, other organic matter. The multi-chambered wall 104 made by plastic film coupled to the storage chamber to enable detection of salt water leakage for repair. chamber to remove remaining undigested organic matter. A floating platform 130 having pump 131 to pump cold water from deep sea by pipe 132, having engine 135, supplied by methane gas by pipe 136 connected to the biogas units gas pipe 116, a sterilization unit with steam source 133 and centrifuge 134 for separating products from fermentation, and crane 137 to help in lifting solid state fermentor for transferring pretreated media to fermentor,

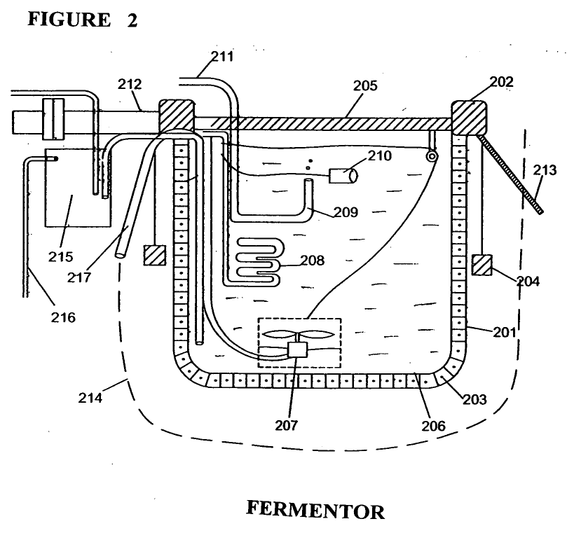

In example embodiment, FIG. 2 illustrates the Fermentor 203, with inner removable layer which is sterilized 206, the fermentor being held by floats 202, the top of the fermentor having a rigid frame with flexible hinged joints and covered by plastic film providing attachment to inlet pipes, pulley of cables to stirrer 207. A aerator pipe 208 is provided, the media with fresh water is feed to the fermentor by channel 212, a camera 210 for monitoring flow and turbidity 210, a suction pipe 217 to transfer spent waste to Bio gas digestor, attachable and detachable weights 204, a vacuum assist to the fermentation process by removing excess alcohol 215 is provided. the whole fermentor is surrounded by a multi chamber jacket 203 with electrodes to detect leakage. The fermentor is also surrounded by a Netting 214 for protection from fish and aquatic animals.

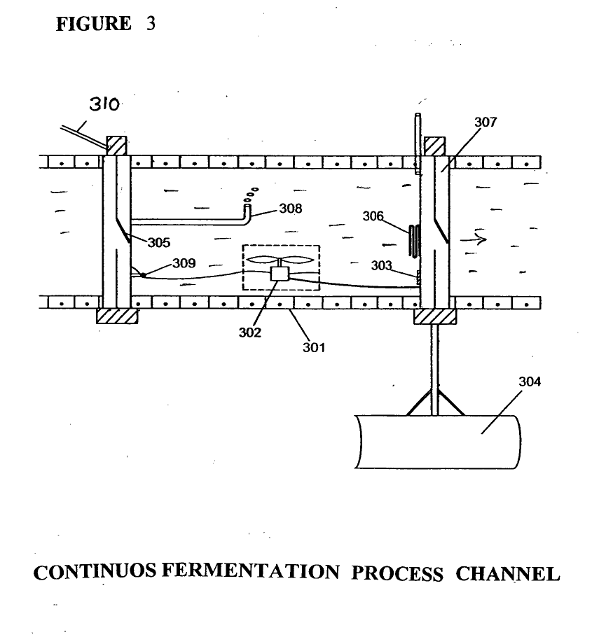

FIG. 3 is a system view of a Continuous flow Fermentor according to one embodiment. Particularly FIG. 3 illustrates multi chamber outer protective layer 301 the Isolator connector 307 which has fixed to it heat exchanger 306, and provided with valve to a opening, a stirrer 302 operated by one of pneumatic motor and one of sealed electric motor and moved by cables wound around pulley 309, provided with an aerator 308 and tow line 310.

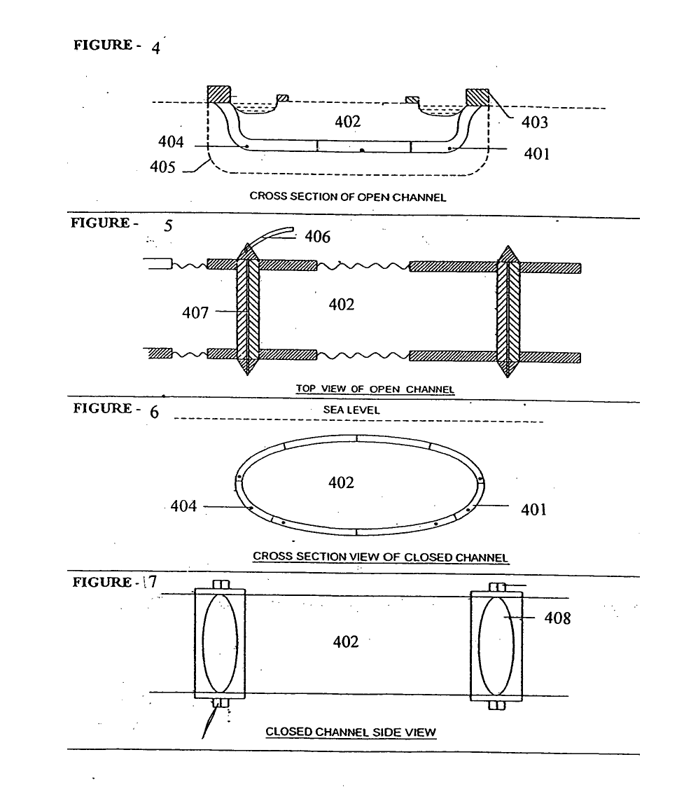

FIG. 4 is a cross section view of the open channel, according to one embodiment. Particularly, FIG. 4 illustrates a leak protection chamber 401, media and fresh water 402, a float 406, electrodes 404, and a netting 405, according to one embodiment.

The leak protection chamber 401 may be used to detecting the leakages of fresh water in the channel (e.g., may be open type, and closed type channel). The media and fresh water 402 may be transferred from the coast. The float 403 (e.g., flexible, and made of plastic film component) may be connected at the end of the flexible film which may be used to float the channels on the surface of the sea. The electrodes 404 attached in the channel is used to sense the salt water leakage in the channel. The netting 405 may be spread over to protect the channel from the fish, and sharks.

In example embodiment, the fermentor that may be connected to channel. The open channel may be held by the float 403 at both the ends of the channel. The netting 405 is used to protect the channel.

FIG. 5 is a top view of the open channel, according to one embodiment. Particularly, FIG. 5 illustrates the float 403, the media fresh water 402, a tow line 406, and a isolator 407, according to one embodiment.

The towing line 406 made of plastic may be used tow the channels, based on error signal to generated through the microprocessor from triangulation signal transmitted by three antennas on the coast for correct designated position of the floating channel. The isolator connector 407 (e.g., may be made of rigid plastic frame with latch) with a drop down valve may be used to connect a channel section with a lift up floats with detachable weights. The isolator connector 407 may also be used to detach, and attached back the floating channel during severe storm, to pass wave energy, to prevent damage to the device, and accidental collusion with ships.

In example embodiment, the top of the isolator connector 407 may be attached with a tow line 406.

FIG. 6 is a cross section view of the closed channel, according to one embodiment. Particularly, FIG. 6 illustrates the leak detection chamber 401, media and fresh water 402, and the electrodes 404, according to one embodiment.

FIG. 6 illustrates another type channel called the closed type channel to collect and transfer the media containing the fresh water 404 along with the electrodes 408 and the leak detection chamber 401 which may be used to detect the salt water leakage in the channel. The functionality of the open type channel and the closed type channel is similar.

FIG. 7 is a side view of the closed channel, according to one embodiment. Particularly, FIG. 7 illustrates the media with fresh water 402, a drop down valve 408, and the latching connector to drop down valve, according to one embodiment.

The drop down valve 408 (e.g., the drop down valve, the butterfly valve, and the flap valve) may be connected to the isolator connector may be directional to take advantage of wave to enable forward flow, and to prevent back flow of the fresh water. There may be drop down valve used during detaching, and for the reattachment of the channel.

FIG. 7 illustrates the drop down valve 408 with latching connector of the isolator of the closed channel.

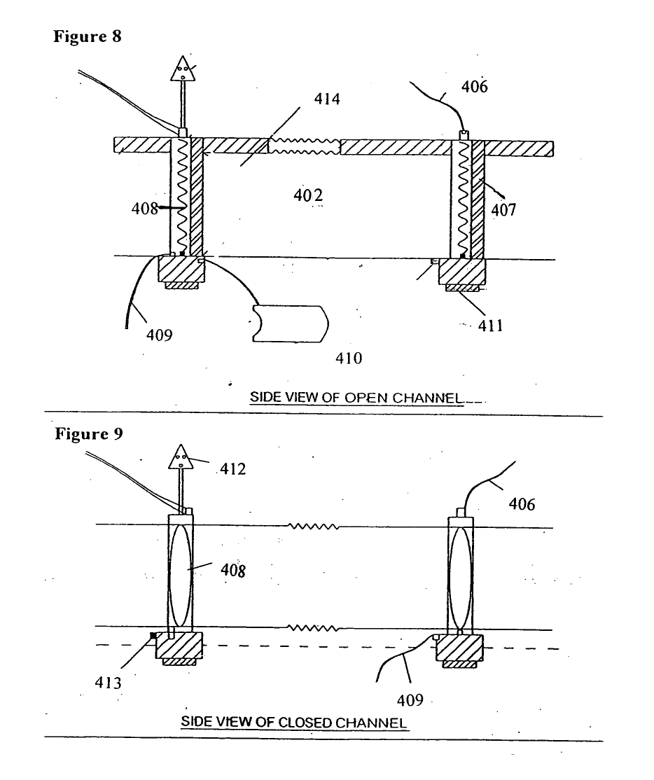

FIG. 8 is a side view of the open channel, according to one embodiment. Particularly, FIG. 8 illustrates the isolator connector 407, media and the fresh water 402, the float 403, the tow line 406, the drop down valve 408, a signal board with blinking LED 412, a damper film structure 410, an anchorage to sea floor 409, and a detachable weight 411, according to one embodiment.

The signal board with blinking LED 412 is carried on the isolator connector to warm ships, and fishing boat from approaching the channel. The damper film structure 410 may be suspended in deeper part of sea, is attached to the Isolator Connector 407. The anchorage to sea floor 409 may be taken wherever necessary by anchor hooks connected to the plastic ropes for stabilization of the floating channel. The detachable weight 411 may be provided to the isolator connector 407 of the floating channel for use during severe storm.

FIG. 8 illustrates a side view of the open channel having isolator connector 407 connected upwardly to the signal board with blinking LED 412, the drop down valve 408. The anchorage to sea floor 409 and the damper film structure 410 are also provided to the isolator connector 407 for stabilization of the device.

FIG. 9 is a side view of the closed channel, according to one embodiment. Particularly, FIG. 9 illustrates the media and fresh water 402, the tow line 406, the drop down valve 408. the signal board with blinking LED 412, and electrodes for shark repulsion 413, according to one embodiment.

The electrodes 413 (e.g., may be stainless steel) attached to the isolator connector 407 downwardly may be used to repel sharks from the device.

FIG. 10 is a cross section view of a towing boat, according to one embodiment. Particularly, FIG. 10 illustrates the tow line 406, an antenna 505A-C, an antenna 504, and a microprocessor 501, according to one embodiment.

The antenna 505A-C may be mounted on the coast to transmit triangulation signal for the corrected designated position of the floating channel. The antenna 504 is mounted in the towing boat to receive signals from coast based transmitter and enable keep the channel in correct position when drifted by waves. The microprocessor 501 may be used to generate error signal that may be used by the towing boat with plastic tow line to place the floating channel in correct designated position when drifted by waves. Motor driven spools having plastic cables enable to provide force for keeping the channels in designated position.

FIG. 10 illustrates the towing boat with plastic tow line along with the antenna 504 and the microprocessors 501 using the error signal generated through the microprocessor 501 from the triangulation signal transmitted by the three antennas on the coast, and by GPS signal tow the channel into correct designated position when drifted by waves.

FIG. 11 is a system view of bellow chamber with isolator to cross ships, according to one embodiment. Particularly, FIG. 11 illustrates a bellow chamber 506. The bellow chamber 506 may be pulled in by a pair of cables that may be driven by motor to allow large ships. The open type, and the closed type channels may have bellow chambers 506.

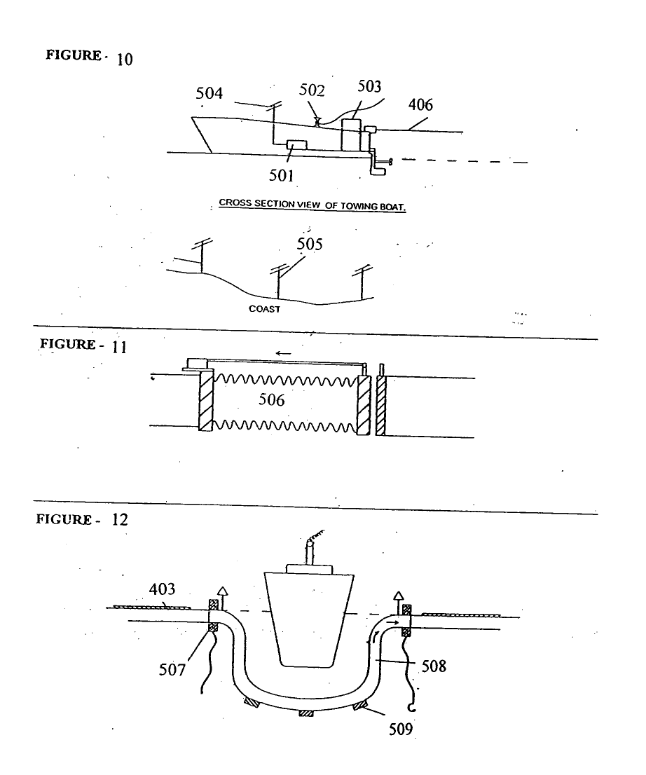

FIG. 12 is a system view of a ‘U’ shaped connectivity chamber, according to one embodiment. Particularly, FIG. 12 illustrates the floating channel having ‘U’ shape connecting chamber 508 (e.g., made of rigid material with swivel joints) may be used to allow the ships to cross the channel without damaging the device. The ‘U’ shaped connecting chamber 508 may be in horizontal position which may dip down to vertical position using the ships force allowing ships to cross and return to horizontal position after the crossing of ships.

Although the present embodiments have been described with reference to specific example embodiments, it will be evident that various modifications and changes may be made to these embodiments without departing from the broader spirit and scope of the various embodiments.

In addition, it will be appreciated that the various operations, processes, and methods disclosed herein may be embodied in a machine-readable medium and/or a machine accessible medium compatible with a data processing system (e.g., a computer system), and may be performed in any order (e.g., including using means for achieving the various operations). Accordingly, the specification and drawings are to be regarded in an illustrative rather than a restrictive sense.

I claim:

1. A device, system and method to produce Alcohol, Bio fuels and other compounds on the sea in large scale in a cost effective way from bulky organic matter, made with low carbon footprint, comprising of:
A Fermentor made of flexible plastic film as separator barrier between sea water and media, held by floats near sea surface, coupled to a inlet channel open and closed type made of flexible plastic film to convey media and fresh water with closed type channel converted into Continuous Fermentation process channel, a solid state fermentor made of flexible plastic film for pretreatment of media, a solar based distillation unit with vacuum support for purifying product by vacuum pump operated by wave and tidal energy, a Bio gas digester made of flexible plastic film as separator between sea water and organic waste matter to be digested to generate methane used as fuel, a Floating platform with engine, pumps, sterilisation unit, centrifuge and microprocessor for control:

2. The devise as in claim 1
wherein Fermentor of large volume at low cost per unit volume, is held by multi-chambered plastic floats embedded with plastic ropes to provide hold for strong anchorage, and stabilization, and the Fermentor to rest on sea bed where it is shallow.
a protection jacket being multi-chambered made from plastic film encloses the Fermentor, to enable detection of leakage for taking up repair, occurrence of leakage is detected through electrodes as sensors;

3. The device as claimed in claim 1:
Wherein the Fermentor is made of at least plastic flexible film with reinforcing fiber thread and polymeric fabric coated with plastic and of rubber sheet reinforced with fiber thread and metal cable for strength, and Wherein the plastic film is made of two layers including a strong thick outer layer made of recycled plastic, and an inner thin layer made of much thinner newly synthesized plastic to give better sealing, the plastic film exposed to sunlight is incorporated with ultraviolet protective chemicals to protect from ultra violet radiation, the chamber is enclosed by the multi-chambered wall, and an extra inner lining thin plastic film is provided inside the digestion chamber to enable separate removal, cleaning and sterilisation. The outside of the chamber is provided a additional layer of thin plastic film covering to manage and clean barnacles where it is a problem.

4. The device as claimed in claim 1:
Wherein the fermentor is provided with a stirring mechanism powered by one of fluid pressure pneumatically and one of sealed electric motor, the stirrer being moved by means of cables pulled by a set of motors, a heat exchanger to maintain required temperature, an aerator pipe to pump air when required, a camera for monitoring flow and turbidity and sensors for process parameters pH, temperature, and other parameters required with provision for taking samples for testing.

5. The device as claimed in claim 1:
Wherein stainless steel electrodes are used in the multi-chambered wall jacket enclosing The fermentor which are connected by at least metallic wire embedded in plastic film and of conductive plastic, to detect salt water leakage which leads to increased electrical conductivity detected by electronic system having microprocessor with dedicated software and thereby enable repair at the needed place.

6. The device as claimed in claim 1:
Wherein the channels made of plastic component is at least one of an open type channel, and a closed type channel used to transfer the media containing fresh water to the fermentor, and wherein the open type channel is formed of at least flexible plastic film reinforced by fiber threads and polymeric fabric coated with plastic and of rubber sheet reinforced with fiber thread and metal cable for strength, held by multi-chambered floats with Isolator Connector being rigid having directional valve, and points for fixing plastic ropes for anchorage, and stabilization, and floats with detachable weights for wave management which are reattached later and till that time held by separate floats, a signal board with blinking light emitting diode and wherein the plastic flexible film surrounded by leakage detection chambers with sensors, and wherein the closed type channels is formed of flexible plastic film reinforced with fiber threads, with Isolator Connector being rigid having directional valve, and points for fixing plastic ropes for anchorage, and stabilization, and floats with detachable weights for wave management which are reattached later and till that time held by separate floats, and wherein the plastic flexible film surrounded by leakage detection chambers with sensors.

7. The device as claimed in claim 1:
wherein the open type, and closed type channel to have ‘U’ shaped connecting chamber made of rigid material with swivel joints, to allow the ships to cross the channel, and wherein the ‘U’ shaped connecting chamber can be in horizontal position which dip down to vertical position using the ships force thereby allowing ships to cross and return to horizontal position after the crossing the ships, and wherein the open type, and closed type channel have Bellow chambers pulled in by a pair of cables driven by motor to allow large ships.

8. The devise as in claim 1
wherein a floating solid state fermentor formed of plastic film with reinforcing fibers for strength with rigid End connector made of plastic material with attachable and detachable weights to enable rotation of the solid state fermentor, wherein the solid state fermentor is provided with plastic matrix to enable dispersion and disintegration of the media after one of fungal growth by forcing freshwater under pressure through pipes provided with holes and where ever the media is such that disintegration is difficult, the end connector is detached after taking the solid state fermentor over the floating Platform and the media removed and transferred to the main fermentor for alcohol production.

9. The device as claimed in claim 1:
wherein a floating solar powered distillation unit comprising a heater one of glass tube having heat exchanger surrounded by vacuum enclosed in a floating plastic container with glass sheet cover on top to trap solar radiation and prevent heat from escaping and one of heat exchanger enclosed in a floating plastic container with glass sheet cover on top to trap solar radiation and prevent heat from escaping, with vapour of alcohol condensing in the floating retort having balancer floats and condenser which is cooled using cold water circulated by pump in a closed circuit so that minimum pumping energy is used. The evaporation is assisted by vacuum derived from pumps operating on tidal and sea waves and from vacuum pump on platform, the solar powered distillation unit is used to generate fresh water by distillation and hot water for generating steam for sterilization.

10. The device as claimed in claim 1:
wherein a Floating Platform having engine and pumps, a sterilization unit with steam generated using hot water from solar distillation unit with supplementary heating using one of bio gas from bio gas generator and a tank for chemical sterilization and UV radiation source, a storage tank for products and media, a centrifuge to separate products from the media, a crane to lift solid state fermentor to remove pretreated media, disintegrate for sending it to Fermentor for breakdown to alcohol, a Microprocessor based control system connected to sensor and camera signal outputs from Fermentor and distillation unit for interpretation of the process and provide signal for actuation of motors and valves by means of dedicated software, and provide a resting place for workers.

11. The device as claimed in claim 1:
Wherein the Bio gas digester is made of at least plastic flexible film with reinforcing fiber thread and polymeric fabric coated with plastic and of rubber sheet reinforced with fiber thread and metal cable for strength, and Wherein the plastic film is made of two layers including a strong thick outer layer made of recycled plastic, and an inner thin layer made of much thinner newly synthesized plastic to give better sealing. The plastic film exposed to sunlight is incorporated with ultraviolet protective chemicals to protect from ultra violet radiation. The chamber is enclosed by the multi-chambered wall, and an extra inner lining thin plastic film is provided inside the digestion chamber to enable separate removal, and cleaning. The outside of the chamber is provided a additional layer of thin plastic film covering to manage and clean barnacles where it is a problem.

12. The device as claimed in claim 1:
Wherein the suction channel is provided to the Bio gas digester to remove remaining organic matter is pumped to the coast through channels connected parallel to the input channels for use as fertilizer.

13. The device as claimed in claim 1:
Wherein the remaining fresh water from BioGas digester is pumped into storage and water treatment unit formed of flexible plastic film reinforced by fiber thread, held by floats for water treatment with at least chlorine, with sedimentation and filtration and then pumped back to coast
